(19) United States
(12) Patent Application Publication
(10) Pub. No.: US 2011/0129939 A1
(43) Pub. Date: Jun. 2, 2011

(54) PORTABLE ELECTRONIC DEVICE AND METHOD FOR USING THE SAME

(75) Inventor: Hsing-Yuan Hsieh, Shindian (TW)

(73) Assignee: FIH (HONG KONG) LIMITED, Kowloon (HK)

(21) Appl. No.: 12/814,559

(22) Filed: Jun. 14, 2010

(30) Foreign Application Priority Data
Nov. 30, 2009 (CN) 200910310678.9

(51) Int. Cl. G01N 33/50; G01N 1/22

(52) U.S. Cl. 436/120; 422/84

(57) ABSTRACT

A portable electronic device includes at least one function module selected from a group consisting of a wireless communication module, an image capturing module, an audio/video file player module, and an internet module, a halitosis measuring module, and a processor connected to the function module and the halitosis measuring module. The processor controls the function module to work, and detects a level of volatile sulfur compounds (VSCs) in a person's mouth using the halitosis measuring module.

### BACKGROUND

1. Technical Field

The present disclosure relates to portable electronic devices, and particularly to a multifunctional portable electronic device with a halitosis measuring module and a method for using the same.

2. Description of Related Art

Volatile sulfur compounds (VSCs) in people's breath may cause halitosis. If a person has serious halitosis it can be very unpleasant to others nearby. Furthermore, serious halitosis may indicate an illness. Therefore, it could be useful to measure a person's halitosis. Since halitosis is commonly caused by VSCs, many halitosis test devices are configured to measure the concentration of VSCs in a person's breath when they exhale.

Generally, conventional halitosis meters only have the single function of testing VSC levels in persons' mouths. If a halitosis meter is integrated with a more frequently used instrument, it may be more convenient and cost effective.

Therefore, there is room for improvement within the art.

### BRIEF DESCRIPTION OF THE DRAWINGS

Many aspects of the present portable electronic device and method for using the same can be better understood with reference to the following drawings. The components in the various drawings are not necessarily drawn to scale, the emphasis instead being placed upon clearly illustrating the principles of the present portable electronic device and method for using the same. Moreover, in the drawings, like reference numerals designate corresponding parts throughout the figures.

FIG. 1 is a block diagram of a portable electronic device, according to an exemplary embodiment.

FIG. 2 is a schematic view of the portable electronic device shown in FIG. 1.

FIG. 3 is a flow chart of a method for using the portable electronic device shown in FIG. 1, according to an exemplary embodiment.

### DETAILED DESCRIPTION

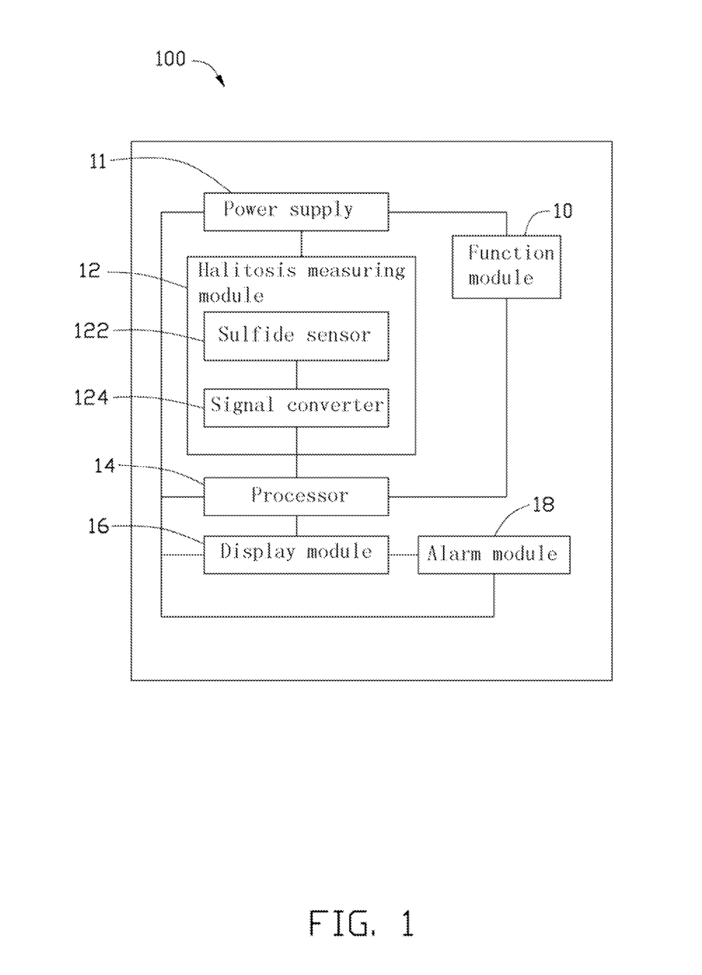

FIG. 1 schematically shows a portable electronic device 100, according to an exemplary embodiment. The device 100 can be a mobile phone, a digital camera, or a personal digital assistant (PDA), for example. The device 100 includes at least one conventional function module 10, such as a wireless communication module, an image capturing module, an audio/video file player module, an internet module, etc., and further includes a power supply 11, a halitosis measuring module 12, a processor 14, a display module 16, and an alarm module 18.

The power supply 11 can be a conventional battery. The power supply 11 is electrically connected to the function module 10, the halitosis measuring module 12, the processor 14, the display module 16, and the alarm module 18 to provide power.

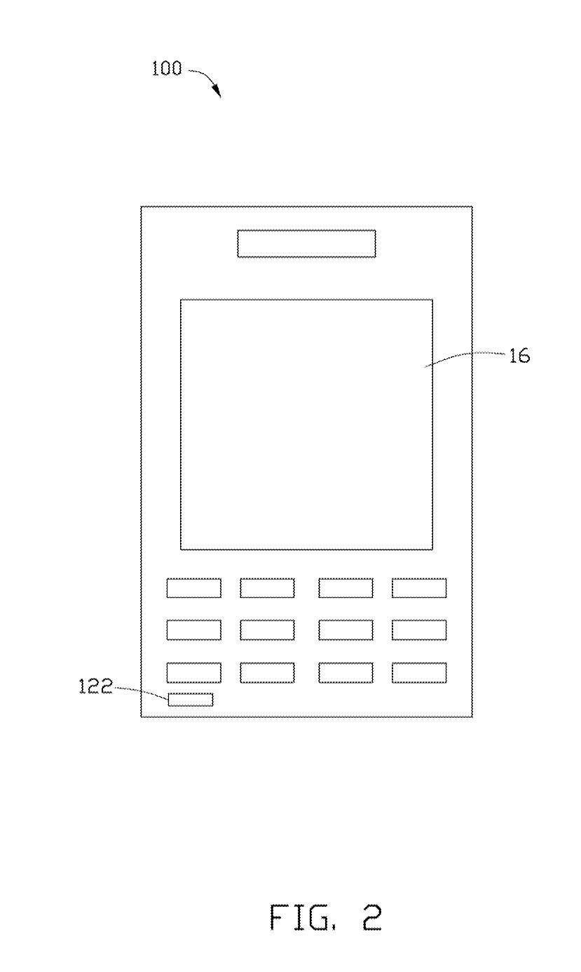

The halitosis detection module 12 includes a sulfide sensor 122 and a signal converter 124. Also referring to FIG. 2, at least a portion of the sulfide sensor 122 is installed on an exterior surface of the portable electronic device 100. When the sulfide sensor 122 is positioned adjacent to the mouth of a person it can detect levels of volatile sulfide compounds (VSCs) in their exhalation. A resistance in the sulfide sensor 122 varies predictably according to the VSC level around the sensor 122 from a tested exhalation.

The signal converter 124 is an analog/digital (A/D) converter. The signal converter 124 is electrically connected to the sulfide sensor 122 and the processor 14. When the power supply 11 applies an electric potential to the halitosis measuring module 12, the signal converter 124 can detect the electric potential on the sulfide sensor 122, convert detected electric potential on the sulfide sensor 122 to digital signals, and transmit the digital signals to the processor 14.

The processor 14 can be a conventional central processing unit (CPU) of the portable electronic device 100. The processor 14 can obtain the electric potential on the sulfide sensor 122 according to the digital signals received from the signal converter 124, and then calculate the resistance of the sulfide sensor 122 according to the electric potential on the sulfide sensor 122. Further, the processor 14 can calculate the VSC level in the person's mouth according to the resistance of the sulfide sensor 122. The processor 17 can also be connected to the function module 10 to control the function module 10 to work.

The display module 16 can be a conventional display of the portable electronic device 100. The alarm module 18 can be a conventional indicator light, bell, or vibrator of the portable electronic device 100. The processor 14 can control the display module 16 and the alarm module 18 to function. When obtaining the VSC level in the person's mouth, the processor 14 can transmit the VSC level data to the display module 16 for display. A limit of VSC level can be set and stored in the processor 14. If the VSC level in the person's mouth exceeds the limit, it indicates that the person has halitosis. Thus, the processor 14 can control the alarm module 18 to give the alarm by controlling operation of lights, an audio device, or a vibration device.

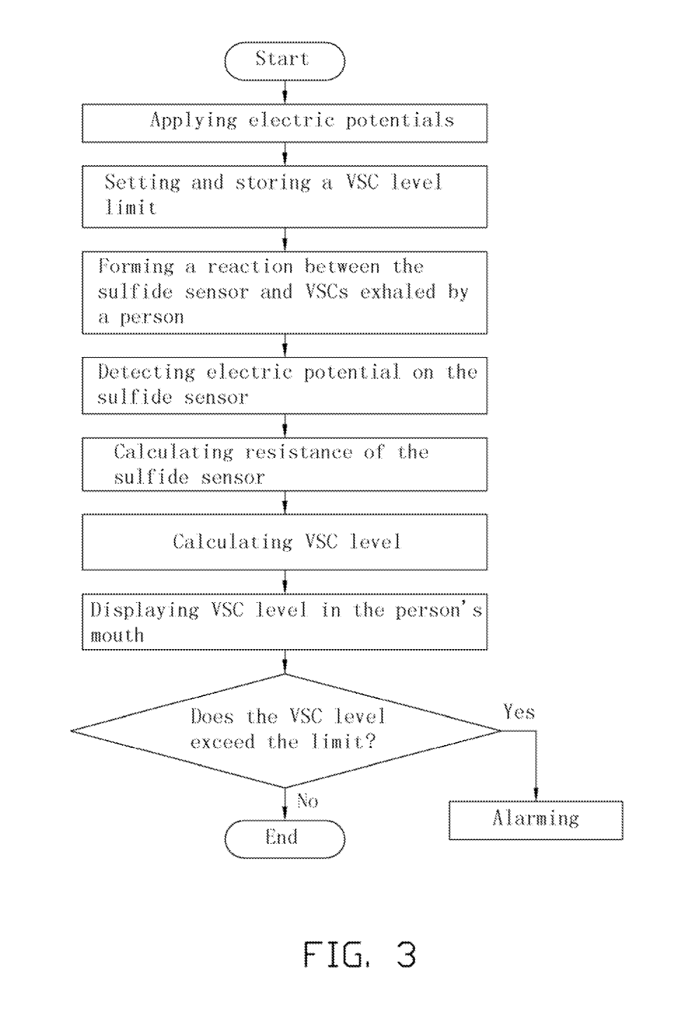

Also referring to FIG. 3, a method for using the portable electronic device 100 to measure the VSC level in a person's mouth, according to an exemplary embodiment, is provided. The method may at least include the following steps.

First, the portable electronic device 100 is actuated, and the power supply 100 applies electric potentials to the halitosis measuring module 12, the processor 14, the display module 16, and the alarm module 18. A limit of VSC level in persons' mouths is set and stored in the processor 14. The portable electronic device 100 is positioned adjacent to a tested person's mouth, and the sulfide sensor 122 is positioned as close to the person's mouth as possible to maintain the accuracy of the halitosis measuring module 12 by preventing the diffusion of gas breathed out by the person.

The tested person blows or speaks, such that the gas breathed out by the person arrives at the sulfide sensor 122. Thus, VSCs in the person's mouth are exhaled with the gas and react with the sulfide sensor 122, and the resistance of the sulfide sensor 122 changes. The signal converter 124 detects current electric potential on the sulfide sensor 122, converts detected electric potential on the sulfide sensor 122 to digital signals, and transmits the digital signals to the processor 14.

When receiving the digital signals corresponding to current electric potential on the sulfide sensor 14, the processor 14 obtains the current electric potential on the sulfide sensor 122 according to the digital signals received from the signal converter 124, and then calculates current resistance of the sulfide sensor 122 according to the electric potential on the sulfide sensor 122. Further, the processor 14 calculates the VSC level in the person's mouth according to the current resistance of the sulfide sensor 122.

When obtaining the VSC level in the person's mouth, the processor 14 transmits the VSC level data to the display module 16 to display. If the VSC level exceeds the limit, then halitosis is indicated. Thus, the processor 14 controls the alarm module 18 to give the alarm, thereby alerting the examiner and the examined person. After the person is tested, the sulfide sensor 122 is positioned in clean air to remove residual VSC from around the sulfide sensor 122. Thus, the original resistance of the sulfide sensor 122 is restored, and the sulfide sensor 122 can be used to detect VSCs level in a next person's mouth.

The present portable electronic device 100 integrated with the halitosis meter device (i.e., the subassembly including at least the power supply 11, the halitosis measuring module 12, the processor 14, the display module 16, and the alarm module 18) is easy to be carried and used. The users of the portable electronic device 100 need not carry a separate halitosis meter device, which can decrease the burden of the users and conserve cost.

It is to be further understood that even though numerous characteristics and advantages of the present embodiments have been set forth in the foregoing description, together with details of structures and functions of various embodiments, the disclosure is illustrative only, and changes may be made in detail, especially in matters of shape, size, and arrangement of parts within the principles of the present invention to the full extent indicated by the broad general meaning of the terms in which the appended claims are expressed.

What is claimed is:

1. A portable electronic device, comprising:
at least one function module selected from a group consisting of a wireless communication module, an image capturing module, an audio/video file player module, and an internet module;
a halitosis measuring module; and
a processor connected to the function module and the halitosis measuring module; wherein the processor controls the function module to work, and detects a level of volatile sulfur compounds (VSCs) in a person's mouth using the halitosis measuring module.

2. The portable electronic device as claimed in claim 1, further comprising a power supply connected to the function module, the halitosis measuring module, and the processor to supply electric potentials to the function module, the halitosis measuring module, and the processor.

3. The portable electronic device as claimed in claim 2, wherein the halitosis measuring module includes a sulfide sensor and a signal converter, the signal converter electrically connected to the sulfide sensor and the processor, the sulfide sensor reacting with VSCs breathed out by the person, and the signal converter detecting electric potential on the sulfide sensor, converting the electric potential on the sulfide sensor to digital signals, and transmitting the digital signals to the processor.

4. The portable electronic device as claimed in claim 3, wherein the processor obtains the electric potential on the sulfide sensor according to the digital signals received from the signal converter, calculates the resistance of the sulfide sensor according to the electric potential on the sulfide sensor, and calculates the VSC level in the person's mouth according to the resistance of the sulfide sensor.

5. The portable electronic device as claimed in claim 2, wherein the reaction between the sulfide sensor and the VSCs breathed out by the person is reversible.

6. The portable electronic device as claimed in claim 1, further comprising a display module connected to the processor to display the VSC level in the person's mouth calculated by the processor.

7. The portable electronic device as claimed in claim 1, further comprising an alarm module connected to the processor to alarm when the VSC level in the person's mouth calculated by the processor exceeds a limit.

8. A method for using a portable electronic device having at least one function module selected from a group consisting of a wireless communication module, an image capturing module, an audio/video file player module, and an internet module and a halitosis measuring module to test a volatile sulfur compounds (VSCs) level in the person's mouth, comprising:
applying an electric potential to a sulfide sensor of the halitosis measuring module;
forming a reaction between the sulfide sensor and VSCs breathed out by the person;
detecting current electric potential on the sulfide sensor;
calculating current resistance of the sulfide sensor according to the current electric potential on the sulfide sensor; and
calculating the VSC level in the person's mouth according to the current resistance of the sulfide sensor.

9. The method as claimed in claim 8, wherein the reaction between the sulfide sensor and the VSCs breathed out by the person is reversible.

10. The method as claimed in claim 8, further comprising activating an alarm when the VSC level in the person's mouth exceeds a limit.
